(19) United States
(12) Patent Application Publication
(10) Pub. No.: US 2010/0210002 A1
(43) Pub. Date: Aug. 19, 2010

(54) Use of plant growth regulators to enhance algae growth for the production of added value products

(76) Inventors: William McCaffrey, Edmonton (CA); Robert Burrell, Edmonton (CA); Mark Burrell, Edmonton (CA); Brett Kotelko, Edmonton (CA)

Correspondence Address: MCCARTER & ENGLISH, LLP BOSTON, 265 Franklin Street, Boston, MA, 02110, US

(21) Appl. No.: 12/684,574

(22) Filed: Jan. 8, 2010

Related U.S. Application Data
(60) Provisional application No. 61/204,920, filed on Jan. 13, 2009.

(51) Int. Cl. C12N 1/00

(52) U.S. Cl. 435/257.1

(57) ABSTRACT

The invention provides methods that enhance the production of biomass from algae that grow autotrophically, heterotrophically, or photoheterotrophically, through the use of plant growth regulators (such as growth hormones, indole acidic acid, etc.) and hormone mimics (phenoxyacetic compounds, etc.). The plant growth regulators or mimics thereof may further increase the proportion of the desired value-added products, such as biodiesel or starch, in the algae culture or the harvested biomass.

### REFERENCE TO RELATED APPLICATION

This application claims the benefit of the filing date under 35 U.S.C. §119(e) to U.S. provisional patent application No. 61/204,920, filed on Jan. 13, 2009, the entire contents of which are incorporated herein by reference.

### BACKGROUND OF THE INVENTION

Algae are one of the most prolific and widespread group of organisms on earth. Over 150,000 species of algae are currently known, and it is likely that more remain to be discovered. For the majority of algal species, the basic identifying characteristics and qualities are known, although there may be some uncertainty about how to classify all the different algal species in the overall taxonomy of life.

Algae (including plant-like forms of many different sizes and colors, diatoms, and cyanobacteria) constitute one of the most important types of life on earth, responsible for most of our atmosphere as well as forming the basis of the food chain for many other forms of life. Entire ecosystems have evolved around algae or symbiotically with algae, and the algal environment includes food sources, predators, viruses, and many other environmental elements that we typically associate with higher forms of life.

Despite the extent and importance of algae, direct human use has been limited. Algae are grown or harvested as food, especially in Asia, often in the form of “seaweed.” They are also widely used to produce various ingredients such as colorants and food additives. Algae have also been used in industrial processes to concentrate and remove heavy metal contamination and remnants of diatoms, known as diatomaceous earth, are used as a filtration medium and for other applications.

Algae can also produce oil, starch, and gas, which can be used in production of diesel fuel, alcohol (e.g. ethanol), and hydrogen or methane gas.

While other biological materials can also yield these fuels, what distinguishes algae are their high productivity and theoretical low cost. Algae can grow from 10 to 100 times faster than other forms of plants. Algae can also be highly prolific in their production of desired oils or starches, in some cases producing as much as 60% of their own weight in these forms. In addition to the benefits of high yield, utilizing algae for bio-products does not compete with agriculture for arable land, requiring neither farmland nor fresh water. Moreover, algae achieve all this with the most basic of inputs, needing in most cases only sunlight, water, air, carbon dioxide and simple nutrients as they are photoautotrophs.

Despite the clear potential benefits of algae as a fuel source, actually achieving this potential has proved frustrating and difficult in the past, for a number of reasons. For example, the conditions for optimal algal cell proliferation are not clearly defined, and they are usually different from those required for optimal production of value-added bioproducts (such as oil/lipid or polysaccharides).

### SUMMARY OF THE INVENTION

The invention provides systems and processes for regulating algal growth using certain plant growth regulators (e.g., growth hormones), for the purpose of, for example, production of value-added bio-products (such as oil or starch).

Thus one aspect of the invention provides a method to increase cell proliferation of algae, comprising culturing the algae in the presence of a plant growth regulator or a mimic thereof to increase algal cell number.

In certain embodiments, algal cell number increases by at least about 5%, 10%, 20%, 50%, 75%, 2-fold, 5-fold, 10-fold, 20-fold, 50-fold, 100-fold, 500-fold, 1000-fold, 10⁴-fold (4 logs), 10⁵-fold (5 logs), 10⁶-fold (6 logs), 10⁷-fold (7 logs), 10⁸-fold (8 logs), 10⁹-fold (9 logs) or more.

In certain embodiments, the rate of algal cell division increases by at least about 5%, 10%, 20%, 50%, 75%, 100%, 200%, 500%, 1,000%, etc. or more.

In certain embodiments, the population doubling time for the algal culture under the instant culture condition is about 0.05-2 days.

In certain embodiments, the plant growth regulator comprises at least one, two, three, four, five or more growth hormones selected from: an Auxin, a Cytokinin, a Gibberellin and/or a mixture thereof. Preferably, the growth hormones include at least one or two from each category/class hormones selected from Auxin, Cytokinin, or Gibberellin.

For example, the Auxin may comprise indole acetic acid (IAA) and/or 1-Naphthaleneacetic acid (NAA). Other Auxin mimics may be 2,4-D; 2,4,5-T; Indole-3-butyric acid (IBA); 2-Methyl-4-chlorophenoxyacetic acid (MCPA); 2-(2-Methyl-4-chlorophenoxy)propionic acids (mecoprop, MCPP); 2-(2,4-Dichlorophenoxy) propionic acid (dichloroprop, 2,4-DP); or (2,4-Dichlorophenoxy)butyric acid (2,4-DB).

In certain embodiments, the Gibberellin comprises GA3.

In certain embodiments, the Cytokinin is an adenine-type cytokinin or a phenylurea-type cytokinin. For example, the adenine-type cytokinin or mimic may comprise kinetin, zeatin, and/or 6-benzylaminopurine, and the phenylurea-type cytokinin may comprise diphenylurea and/or thidiazuron (TDZ).

In certain embodiments, the plant growth regulator further comprises vitamin B1 or analog/mimics thereof.

In certain embodiments, only one of the subject growth regulators (e.g., an Auxin family growth regulator or a Cytokinin family growth regulator) is used for algae growth.

In certain embodiments, more than one subject growth regulators are used. In certain embodiments, at least one Auxin family growth regulator and at least one Cytokinin family growth regulator are used, and the weight ratio of the at least one Auxin to at least one Cytokinin is about 1:2 to 2:1 (w/w/), preferably about 1:1 (w/w). In certain embodiments, the ratio (w/w) of Auxin to Gibberellin is about 1:2-2:1, preferably about 1:1. In certain embodiments, the ratio (w/w) of Auxin to vitamin B1 is about 1:4-1:1, preferably about 1:2.

In certain embodiments, the mimic is a phenoxyacetic compound.

In certain embodiments, the method further comprises culturing the algae in a medium with non-limiting levels of nutrients and trace elements required for optimal cell proliferation.

In certain embodiments, the nutrients include one or more C, N, P, S, and/or O sources. Preferably, the concentration of the nutrients are non-toxic for cell division and/or growth.

In certain embodiments, the medium may comprise a liquid separation of an anaerobic biodigestate, optionally supplemented with additional nutrients when and as needed. The anaerobic biodigestate may result from anaerobic digestion of animal offal, livestock manure, food processing waste, municipal waste water, thin stillage, distiller's grains, or other organic materials.

In certain embodiments, the concentrations of the nutrients are non-toxic for cell division and/or growth.

In certain embodiments, the algae are cultured under optimal temperature for cell division, the optimal temperature being in the range of about 0-40° C. for non-thermophilic algae, and about 40-95° C., or about 60-80° C. for thermophilic algae.

In certain embodiments, the algae are cultured in a bio-reactor. Preferably, the bioreactor is adapted for optimal cell proliferation. Preferably, the bio-reactor can be sterilized.

In certain embodiments, the algae metabolize using heterotrophic, photoheterotrophic, or autotrophic physiological mechanisms.

In certain embodiments, the algae are Chromophytes, preferably Chlorophytes or Bacillariophytes. In certain embodiments, the algae are Chlorella sp. (such as Chlorella vulgaris), Auxenochlorella sp. (Auxenochlorella protothecoides), Scenedesmus sp. and Ankistrodesmus sp, etc. In certain embodiments, the algae have frustule free forms. In certain embodiments, the algae is not brown algae (Phaeophyceae) or red algae. In certain embodiments, the algae are not Thraustochytriales.

Another aspect of the invention provides a method to produce an algal product, comprising culturing algae in the presence of a plant growth regulator or a mimic thereof to accumulate the algal product.

In certain embodiments, algal cell number increases by no more than about 1,000%, 300%, 200%, 100%, or 50%.

In certain embodiments, algal biomass substantially increases. For example, in certain embodiments, algal biomass increases by at least about 5%, 10%, 20%, 40%, 60%, 80%, 100%, 150%, 200%. In certain embodiments, algal biomass increases largely as a result of accumulating said algal product.

In certain embodiments, the algae are cultured in a nitrogen-limited medium or a medium with a nitrogen level optimized for algal product synthesis.

In certain embodiments, the plant growth regulator comprises an oil stimulating factor. For example, the oil stimulating factor may comprise a humate, such as fulvic acid or humic acid.

In certain embodiments, the algae are cultured in a bio-reactor. Preferably, the bioreactor is adapted for optimal production of the algal product.

In certain embodiments, the algal product is oil or lipid, such as an algal product comprising Omega-3, -6, and/or -9.

In certain embodiments, the algal product is starch (or a polysaccharide). When starch or polysaccharide is the desired algal product, the algae are preferably not subjected to nitrogen-limitation growing conditions.

Another aspect of the invention provides a system adapted for the algae growing process of the invention. Preferably, the bioreactor can be sterilized to facilitate axenic algal growth under heterotrophic and photoheterotrophic conditions.

It is contemplated that all embodiments described herein can be combined with features in other embodiments wherever applicable.

### BRIEF DESCRIPTION OF THE DRAWINGS

FIG. 1 shows control Chlorella vulgaris grown in Bristol's medium amended with 0.1% yeast extract and 0.5% glucose for seven days.

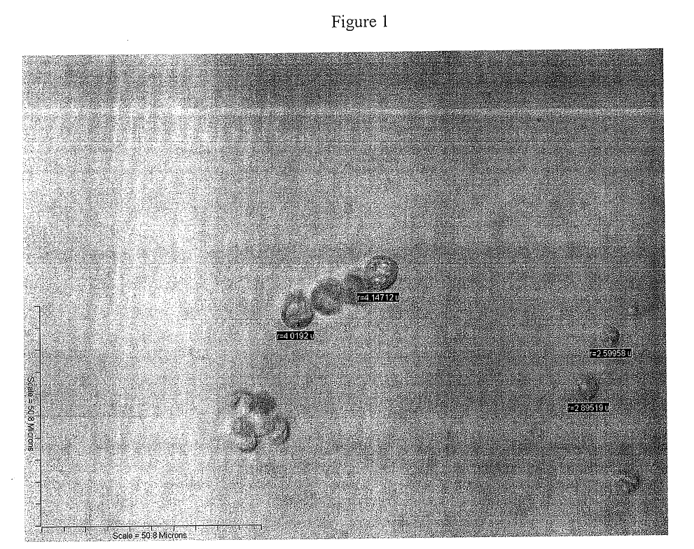

FIG. 2 shows Chlorella vulgaris grown in Bristol's medium amended with 0.1% yeast extract, 0.5% glucose and fulvic acid for seven days.

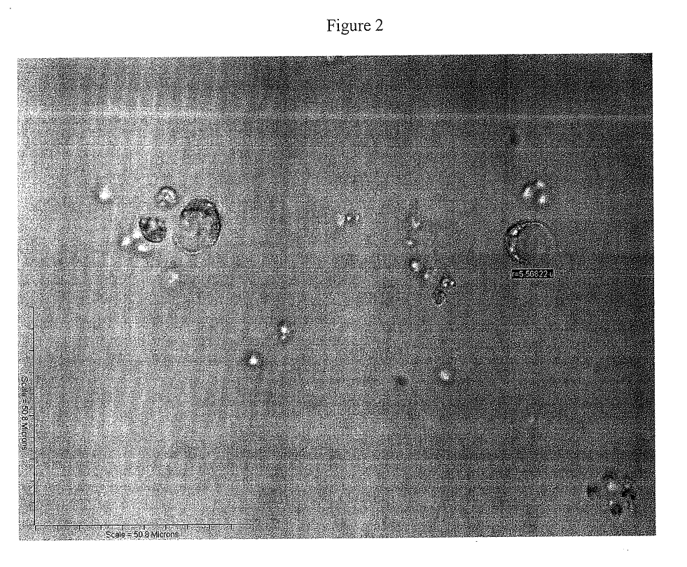

FIG. 3 shows an exemplary growth curve of Chlorella protothecoides in the presence or absence of a combination of plant growth regulators.

FIG. 4 shows an exemplary growth curve of Chlorella protothecoides in the presence or absence of a combination of plant growth regulators.

FIG. 5 shows an exemplary growth curve of Chlorella protothecoides in the presence or absence of a combination of plant growth regulators.

FIG. 6 shows an exemplary growth curve of Chlorella protothecoides in the presence or absence of a combination of plant growth regulators.

### DETAILED DESCRIPTION OF THE INVENTION

One aspect of the invention is partly based on the discovery that algal growth (e.g., cell proliferation during, for example, exponential growth stage or post-exponential growth stage) can be stimulated by certain plant growth regulators or a mimic thereof.

Thus one aspect of the invention provides a method to increase cell proliferation of algae, comprising culturing the algae in the presence of a plant growth regulator or a mimic thereof to increase algal cell number.

Plant hormones or regulators affect gene expression and transcription levels, cellular division, and growth in plants. A large number of related chemical compounds are synthesized by human, and have been used to regulate the growth of cultivated plants, weeds, and in vitro-grown plants and plant cells. These man-made compounds are sometimes called Plant Growth Regulators or PGRs for short. For the synthesized regulator, it may be identical to a naturally occurring regulator, or it may contain chemical modifications not found in nature. “Growth hormones (or mimics thereof)” as used herein includes both natural plant hormones and the man-made/synthetic regulators, mimics, or derivatives thereof. Preferably, the growth hormones/regulators, or mimics thereof, stimulates algal growth at least under one concentration, preferably under a condition similar or identical to the one used in the examples below, such as Examples 3-7. The terms “growth hormone” and “growth regulator” may be used interchangeably herein.

In general, plant hormones and regulators are categorized into five major classes, some of which are made up of many different chemicals that can vary in structure from one plant to the next. The chemicals are each grouped together into one of these classes based on their structural similarities and on their effects on plant physiology. Other plant hormones and growth regulators are not easily grouped into these classes. Rather, they exist naturally or are synthesized by humans or other organisms, including chemicals that inhibit plant growth or interrupt the physiological processes within plants.

The five major classes are: Abscisic acid (also called ABA); Auxins; Cytokinins; Ethylene; and Gibberellins. Other identified plant growth regulators include: Brassinolides (plant steroids that are chemically similar to animal steroid hormones. They promote cell elongation and cell division, differentiation of xylem tissues, and inhibit leaf abscission); Salicylic acid (activates genes in some plants that produce chemicals that aid in the defense against pathogenic invaders); Jasmonates (produced from fatty acids and seem to promote the production of defense proteins that are used to fend off invading organisms. They are also believed to have a role in seed germination, and affect the storage of protein in seeds, and seem to affect root growth); Plant peptide hormones (encompasses all small secreted peptides that are involved in cell-to-cell signaling. These small peptide hormones play crucial roles in plant growth and development, including defense mechanisms, the control of cell division and expansion, and pollen self-incompatibility); Polyamines (strongly basic molecules with low molecular weight that have been found in all organisms studied thus far. They are essential for plant growth and development and affect the process of mitosis and meiosis); Nitric oxide (NO) (serves as signal in hormonal and defense responses); Strigolactones (implicated in the inhibition of shoot branching).

The abscisic acid class of PGR is composed of one chemical compound normally produced in the leaves of plants, originating from chloroplasts, especially when plants are under stress. In general, it acts as an inhibitory chemical compound that affects bud growth, seed and bud dormancy.

Auxins are compounds that positively influence cell enlargement, bud formation and root initiation. They also promote the production of other hormones and in conjunction with cytokinins, they control the growth of stems, roots, and fruits, and convert stems into flowers. Auxins affect cell elongation by altering cell wall plasticity. Auxins decrease in light and increase where it is dark. Auxins are toxic to plants in large concentrations; they are most toxic to dicots and less so to monocots. Because of this property, synthetic auxin herbicides including 2,4-D and 2,4,5-T have been developed and used for weed control. Auxins, especially 1-Naphthaleneacetic acid (NAA) and Indole-3-butyric acid (IBA), are also commonly applied to stimulate root growth when taking cuttings of plants. The most common auxin found in plants is indoleacetic acid or IAA.

An important member of the auxin family is indole-3-acetic acid (IAA). It generates the majority of auxin effects in intact plants, and is the most potent native auxin. However, molecules of IAA are chemically labile in aqueous solution. Other naturally-occurring auxins include 4-chloro-indoleacetic acid, phenylacetic acid (PAA) and indole-3-butyric acid (IBA). Common synthetic auxin analogs include 1-naphthaleneacetic acid (NAA), 2,4-dichlorophenoxyacetic acid (2,4-D), and others. Several exemplary (non-limiting) natural and synthetic auxins that may be used in the instant invention are shown below.

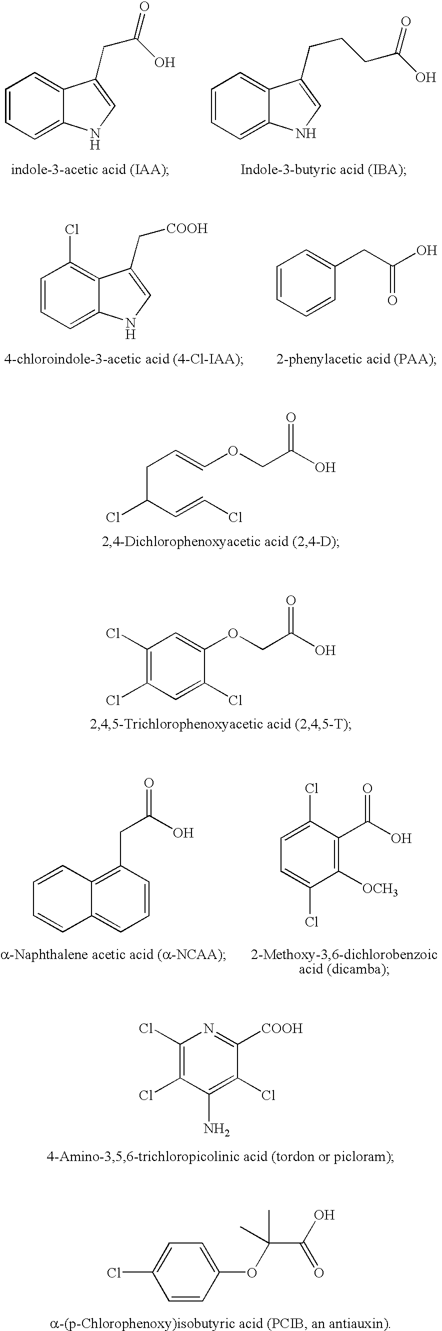

Cytokinins or CKs are a group of chemicals that influence cell division and shoot formation. They also help delay senescence or the aging of tissues, are responsible for mediating auxin transport throughout the plant, and affect internodal length and leaf growth. They have a highly-synergistic effect in concert with auxins and the ratios of these two groups of plant hormones affect most major growth periods during a plant's lifetime. Cytokinins counter the apical dominance induced by auxins; they in conjunction with ethylene promote abscission of leaves, flower parts and fruits.

There are two types of cytokinins: adenine-type cytokinins represented by kinetin, zeatin and 6-benzylaminopurine, as well as phenylurea-type cytokinins like diphenylurea or thidiazuron (TDZ).

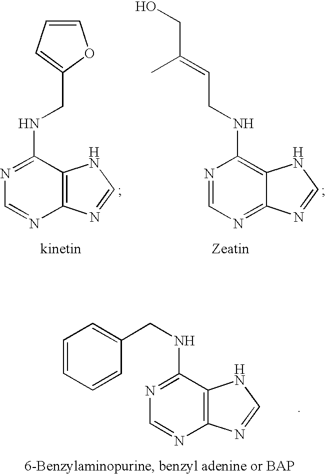

Ethylene is a gas that forms through the Yang Cycle from the breakdown of methionine, which is in all cells. Its effectiveness as a plant hormone is dependent on its rate of production versus its rate of escaping into the atmosphere. Ethylene is produced at a faster rate in rapidly-growing and -dividing cells, especially in darkness. New growth and newly-germinated seedlings produce more ethylene than can escape the plant, which leads to elevated amounts of ethylene, inhibiting leaf expansion. As the new shoot is exposed to light, reactions by phytochrome in the plant's cells produce a signal for ethylene production to decrease, allowing leaf expansion. Ethylene affects cell growth and cell shape; when a growing shoot hits an obstacle while underground, ethylene production greatly increases, preventing cell elongation and causing the stem to swell. The resulting thicker stem can exert more pressure against the object impeding its path to the surface. If the shoot does not reach the surface and the ethylene stimulus becomes prolonged, it affects the stems natural geotropic response, which is to grow upright, allowing it to grow around an object. Studies seem to indicate that ethylene affects stem diameter and height: When stems of trees are subjected to wind, causing lateral stress, greater ethylene production occurs, resulting in thicker, more sturdy tree trunks and branches. Ethylene affects fruit-ripening: Normally, when the seeds are mature, ethylene production increases and builds-up within the fruit, resulting in a climacteric event just before seed dispersal. The nuclear protein ETHYLENE INSENSITIVE2 (EIN2) is regulated by ethylene production, and, in turn, regulates other hormones including ABA and stress hormones.

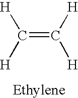

Gibberellins or GAs include a large range of chemicals that are produced naturally within plants and by fungi. Gibberellins are important in seed germination, affecting enzyme production that mobilizes food production used for growth of new cells. This is done by modulating chromosomal transcription. In grain (rice, wheat, corn, etc.) seeds, a layer of cells called the aleurone layer wraps around the endosperm tissue. Absorption of water by the seed causes production of GA. The GA is transported to the aleurone layer, which responds by producing enzymes that break down stored food reserves within the endosperm, which are utilized by the growing seedling. GAs produce bolting of rosette-forming plants, increasing internodal length. They promote flowering, cellular division, and in seeds growth after germination. Gibberellins also reverse the inhibition of shoot growth and dormancy induced by ABA.

All known gibberellins are diterpenoid acids that are synthesized by the terpenoid pathway in plastids and then modified in the endoplasmic reticulum and cytosol until they reach their biologically-active form. All gibberellins are derived from the ent-gibberellane skeleton, but are synthesised via ent-kaurene. The gibberellins are named GA1 . . . GAn in order of discovery. Gibberellic acid, which was the first gibberellin to be structurally characterised, is GA3. As of 2003, there were 126 GAs identified from plants, fungi, and bacteria. Gibberellins are tetracyclic diterpene acids. There are two classes based on the presence of 19 carbons or 20 carbons. The 19-carbon gibberellins, such as gibberellic acid, have lost carbon 20 and, in place, possess a five-member lactone bridge that links carbons 4 and 10. The 19-carbon forms are, in general, the biologically active forms of gibberellins. Hydroxylation also has a great effect on the biological activity of the gibberellin. In general, the most biologically active compounds are dihydroxylated gibberellins, which possess hydroxyl groups on both carbon 3 and carbon 13. Gibberellic acid is a dihydroxylated gibberellin. Representative (non-limiting) gibberellins are shown below:

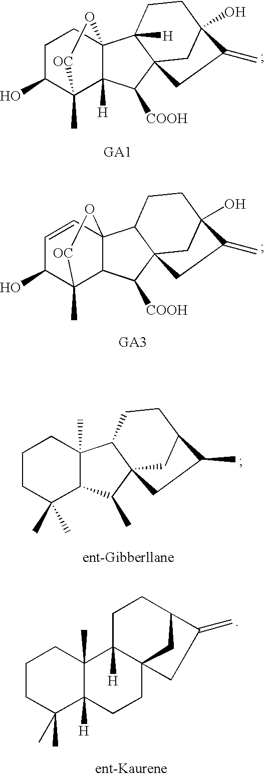

Exemplary growth hormones/regulators or mimics thereof that may be used in the instant invention (e.g., added to the algal culture to boost cell division or proliferation) include those in the Auxin family, the Cytokinin family, and/or the Gibberellin family.

For example, Auxins and mimics useful for the invention include (without limitation): an indole acetic acid (IAA); 2,4-D; 2,4,5-T; 1-Naphthaleneacetic acid (NAA); Indole-3-butyric acid (IBA); 2-Methyl-4-chlorophenoxyacetic acid (MCPA); 2-(2-Methyl-4-chlorophenoxy)propionic acids (mecoprop, MCPP); 2-(2,4-Dichlorophenoxy)propionic acid (dichloroprop, 2,4-DP); (2,4-Dichlorophenoxy)butyric acid (2,4-DB); 4-chloro-indoleacetic acid (4-Cl-IAA); phenylacetic acid (PAA); 2-Methoxy-3,6-dichlorobenzoic acid (dicamba); 4-Amino-3,5,6-trichloropicolinic acid (tordon or picloram); α-(p-Chlorophenoxy)isobutyric acid (PCIB, an antiauxin), or mixtures thereof. When used as a mixture, the mixture preferably has equivalent biological activity (e.g., under substantially the same growth conditions, stimulates algal cell growth to substantially the same extent, preferably in substantially the same amount of time) as an effective amount of IAA (when used alone) or an effective amount of IAA+NAA. See, for example, the conditions used in the examples below.

Cytokinins and mimics useful for the invention may be of an adenine-type or a phenylurea-type, and may include (without limitation) kinetin, zeatin, 6-benzylaminopurine (6-BA or 6-BAP), diphenylurea, thidiazuron (TDZ), or mixtures thereof. Preferably, the adenine-type cytokinins, such as kinetin, zeatin, 6-benzylaminopurine (6-BA or 6-BAP), or mixture thereof, are used. When used as a mixture, the mixture preferably has equivalent biological activity (e.g., under substantially the same growth conditions, stimulates algal cell growth to substantially the same extent, preferably in substantially the same amount of time) as an effective amount of kinetin+6-BA. See, for example, the conditions used in the examples below.

Gibberellins and mimics useful for the invention may be any of the Gibberellins described herein or known in the art, such as GA3. Preferably, the Gibberellins, mimics or derivatives, or mixtures thereof has equivalent biological activity (e.g., under substantially the same growth conditions, stimulates algal cell growth to substantially the same extent, preferably in substantially the same amount of time) as an effective amount of GA3. See, for example, the conditions used in the examples below.

The mimics may also be a phenoxyacetic compound.

To achieve optimal growth stimulatory effect, in certain embodiments, only one of the subject growth regulators (e.g., an Auxin family growth regulator, a Cytokinin family growth regulator, or a Gibberellin family growth factor) is used for algae growth. In certain other embodiments, more than one subject growth regulators are used. For example, at least one Auxin family growth regulator and at least one Cytokinin family growth regulator may be used, and the (weight) ratio of total Auxin to total Cytokinin in the medium may be adjusted to be around 1:2 to 2:1, preferably around 1:1.

Preferably, when Gibberellins are present, the (weight) ration of total Auxin to total Gibberellin in the medium may be adjusted to be around 1:2 to 2:1, preferably around 1:1.

In certain embodiments, vitamin B1 or its mimics, derivatives, or functional equivalents may be present. Preferably, the (weight) ratio of total Auxin to total vitamin B1 in the medium may be adjusted to be around 1:4 to 1:1, preferably around 1:2.

In certain embodiments, the total concentration of the Auxins in the growth medium is about 0.01-0.04 μg/L, about 0.003-0.12 μg/L, about 0.002-0.2 μg/L, or about 0.001-0.4 μg/L.

In certain embodiments, the total concentration of the Cytokinins in the growth medium is about 0.01-0.04 μg/L, about 0.003-0.12 μg/L, about 0.002-0.2 μg/L, or about 0.001-0.4 μg/L.

In certain embodiments, the total concentration of the Gibberellins in the growth medium is about 0.01-0.04 μg/L, about 0.003-0.12 μg/L, about 0.002-0.2 μg/L, or about 0.001-0.4 μg/L.

In certain embodiments, the total concentration of the vitamin B1 compounds in the growth medium is about 0.02-0.08 μg/L, about 0.006-0.24 μg/L, about 0.004-0.4 μg/L, or about 0.002-0.8 μg/L.

In certain embodiments, ethylene, Brassinolides, Salicylic acid, Jasmonates, Plant peptide hormones, Polyamines, Nitric oxide, and/or Strigolactones may be used.

In certain embodiments, ethylene, Brassinolides, Jasmonates, Plant peptide hormones, and/or Polyamines may be used.

In certain embodiments, the presence of one or more hormones/regulators increases algae proliferation by about 15% (e.g., 1.4 to 1.6), 20%, 25%, 30%, 35% or more, preferably under one of the growth conditions in the examples, e.g., Examples 3-7.

According to this aspect of the invention, algal cell number may increase by at least about 5%, 10%, 15%, 20%, 50%, 75%, 2-fold, 5-fold, 10-fold, 20-fold, 50-fold, 100-fold, 500-fold, 1000-fold, 10⁴-fold (4 logs), 10⁵-fold (5 logs), 10⁶-fold (6 logs), 10⁷-fold (7 logs), 10⁸-fold (8 logs), 10⁹-fold (9 logs), or more.

Regardless of the specific plant growth regulators used in the medium, a variety of different media may be used to support algae growth. Generally, a suitable medium may contain nitrogen, inorganic salts of trace metal (e.g., phosphorous, potassium, magnesium, and iron, etc.), vitamins (e.g., thiamine), and the like, which may be essential to growth. For example, media such as the VT medium, C medium, MC medium, MBM medium, and MDM medium (see Sorui Kenkyuho, ed. by Mitsuo Chihara and Kazutoshi Nishizawa, Kyoritsu Shuppan (1979)), the OHM medium (see Fabregas et al., J. Biotech., Vol. 89, pp. 65-71 (2001)), the BG-11 medium, Bristol's medium, and modifications thereof may be used. Other examples of suitable media include, but are not limited to, Luria Broth, brackish water, water having nutrients added, dairy runoff, media with salinity of less than or equal to 1%, media with salinity of greater than 1%, media with salinity of greater than 2%, media with salinity of greater than 3%, media with salinity of greater than 4%, and combinations thereof. The most preferred medium include a liquid separation of an anaerobic biodigestate, optionally supplemented with additional nutrients. The liquid may be separated from the anaerobic biodigestate by mechanical means, such as by using a screw press or by centrifugation. The liquid ideally comprise no more than 5-10% solid content, preferably no more than 8% solid content.

These media may be selected depending on their purposes, such as growth or proliferation, or induction of the desired algal product. For example, for optimal cell division/proliferation, a medium having a large amount of components serving as a nitrogen source is used (e.g., rich medium: containing at least about 0.15 g/L expressed in terms of nitrogen). For algal product production (e.g., oil), a medium having a small amount of components serving as a nitrogen source is preferred (e.g., containing less than about 0.02 g/L expressed in terms of nitrogen). Alternatively, a medium containing a nitrogen source at an intermediate concentration between these media may be used (low nutrient medium: containing at least 0.02 g/L and less than 0.15 g/L expressed in terms of nitrogen).

In other words, during the exponential growth stage, the medium preferably has non-limiting levels of nutrients (including one or more C, N, P, S, and/or O sources) and trace elements required for optimal cell number increase. Preferably, the concentrations of the nutrients are non-toxic for cell division and/or growth.

The nitrogen concentration, phosphorous concentration, and other properties of the medium can be determined depending on the amount of the algae to be inoculated and their expected growth rate. For example, when an algal count in the order of 10⁵ cells per milliliter is inoculated in a low nutrient (e.g. nitrogen) medium, the algae will grow to a certain extent, but the growth will stop because the amount of the nitrogen source is too small. Such a low nutrient medium is suitable for performing growth and algal product production continuously in a single step (e.g., in a batch manner). Furthermore, by adjusting the N/P mole ratio to value from about 10-30, preferably 15-25, or by adjusting the C/N mole ratio to value from about 12-80 (e.g., a lower N content), the alga can be induced to produce the desired bio-product (e.g., oil). In the case where the algae count for inoculation is higher, the rich medium can be employed to perform the above-described cultivation. In this manner, the composition of the medium can be determined in consideration of various conditions.

Nitrogen sources or nitrogen supplements in the algal growth media can include nitrates, ammonia, urea, nitrites, ammonium salts, ammonium hydroxide, ammonium nitrate, monosodium glutamate, soluble proteins, insoluble proteins, hydrolyzed proteins, animal by-products, dairy waste, casein, whey, hydrolyzed casein, hydrolyzed whey, soybean products, hydrolyzed soybean products, yeast, hydrolyzed yeast, corn steep liquor, corn steep water, corn steep solids, distillers grains, yeast extract, oxides of nitrogen, N₂O, or other suitable sources (e.g., other peptides, oligopeptides, and amino acids, etc.). Carbon sources or carbon supplements can include sugars, monosaccharides, disaccharides, sugar alcohols, fats, fatty acids, phospholipids, fatty alcohols, esters, oligosaccharides, polysaccharides, mixed saccharides, glycerol, carbon dioxide, carbon monoxide, starch, hydrolyzed starch, or other suitable sources (e.g., other 5-carbon sugars, etc.).

Additional media ingredients or supplements can include buffers, minerals, growth factors, anti-foam, acids, bases, antibiotics, surfactants, or materials to inhibit growth of undesirable cells.

The nutrients can be added all at the beginning, or some at the beginning and some during the course of the growing process as a single subsequent addition, as a continuous feed during algal growth, as multiple dosing of the same or different nutrients during the course of the growth, or as a combination of these methods.

The pH of the culture, if desired, can be controlled or adjusted through the use of a buffer or by addition of an acid or base at the beginning or during the course of the growth. In some cases, both an acid and a base can be used in different zones of the reactor or in the same zone at the same or different times in order to achieve a desirable degree of control over the pH. Non-limiting examples of buffer systems include mono-, di-, or tri-basic phosphate, TRIS, TAPS, bicine, tricine, HEPES, TES, MOPS, PIPES, cacodylate, MES, and acetate. Non-limiting examples of acids include sulfuric acid, HCl, lactic acid, and acetic acid. Non-limiting examples of bases include potassium hydroxide, sodium hydroxide, ammonium hydroxide, ammonia, sodium bicarbonate, calcium hydroxide, and sodium carbonate. Some of these acids and bases in addition to modifying the pH can also serve as a nutrient for the cells. The pH of the culture can be controlled to approximate a constant value throughout the entire course of the growth, or it can be changed during the growth. Such changes can be used to initiate or end different molecular pathways, to force production of one particular product, to force accumulation of a product such as fats, dyes, or bioactive compounds, to suppress growth of other micro-organisms, to suppress or encourage foam production, to force the cells into dormancy, to revive them from dormancy, or for some other purposes.

In certain embodiments, it is preferable that the pH is maintained at about 4-10, or about 6 to 8 throughout the cultivation period.

Likewise, the temperature of the culture can in some embodiments be controlled or adjusted to approximate a particular value, or it can be changed during the course of the growth for the same or different purposes as listed for pH changes. For example, optimal temperature for cell division may be in the range of about 0-40° C., 20-40° C., 15-35° C., or about 20-25° C. for non-thermophilic algae; and about 40-95° C., preferably about 60-80° C. for thermophilic algae.

In certain of such embodiments, a temperature control component is provided that comprises a temperature measurement component that measures a temperature within the system, such as a temperature of the medium, and a control component that can control the temperature in response to the measurement. The control component may comprise a submerged coil or a jacket on the side or bottom wall of the culture container.

The algae may be cultured in a natural environment, such as an open pond, channel, or trench, etc., or in a closed bioreactor (container or vessel, etc.). If growing condition needs to be changed or adjusted, the algae culture may be grown in a first bioreactor under the first growing condition, and in a second bioreactor under the second growing condition, etc. The different steps may be performed independently in a batch manner using separate culture tanks/vessels. It is also possible to wash and collect the grown algae at the end of the one step, place the algae back in the same culture tank, and then perform the next step. In certain embodiments, washing is optional, and may or may not be necessary depending on the medium in the first reactor.

Open ponds (or channels, etc.) or closed (preferably sterilizable) bioreactors can be operated in batch mode, continuous mode, or semi-continuous mode. For example, in a batch mode, the pond/bioreactor would be filled to appropriate level with fresh and/or recycled media and inoculums. This culture would then be allowed to grow until the desired degree of growth has occurred. At this point, harvest of the product would occur. In one embodiment, the entire pond/bioreactor contents would be harvested, then the pond/bioreactor can be cleaned and sanitized (e.g., sterilized for the bioreactor) as needed, and refilled with media and inoculums. In another embodiment, only a portion of the contents would be harvested, for example approximately 50%, then media would be added to refill the pond/bioreactor and the growth would continue.

Alternatively, in a continuous mode, media, fresh and/or recycled, and fresh inoculums are continuously fed to the pond/bioreactor while harvest of cellular material occurs continuously. In continuous operation, there can be an initial start-up phase where the harvest is delayed to allow sufficient cell concentration to build up. During this start-up phase, the media feed and/or inoculums feed can be interrupted. Alternatively, media and inoculums can be added to the pond/bioreactor and when the pond/bioreactor gets to the desired liquid volume, harvest commences. Other start-up techniques can be used as desired to meet operational requirements and as appropriate for the particular product organism and growth medium. Where a culture is grown in a first pond/bioreactor, approximately 10-90%, or 20-80%, or 30-70% of the culture may be transferred to a second pond/bioreactor, with the residual contents serving a starter culture for subsequent growth in the first pond/bioreactor. Alternatively, about 100% of the culture is transferred to the second pond/bioreactor, while the first pond/bioreactor is inoculated from a new source.

A continuous pond/bioreactor culture can be operated in a “stirred mode” or a “plug flow mode” or a “combination mode.” In a stirred mode, the media and inoculums are added and mixed into the general volume of the pond/bioreactor. Mixing devices include, but are not limited to paddlewheel, propeller, turbine, paddle, or airlift operating in a vertical, horizontal or combined direction. In some embodiments, the mixing can be achieved or assisted by the turbulence created by adding the media or inoculums. The concentration of cells and media components does not vary greatly across the horizontal area of the pond/bioreactor. In a plug flow mode, the media and inoculums are added at one end of the pond/bioreactor, and harvest occurs at the other end. In the plug flow mode, the culture moves generally from the media inlet toward the harvest point. Cell growth occurs as the culture moves from the inlet to the harvest location. Movement of the culture can be achieved through means including, but not limited to, sloping the pond/bioreactor, mixing devices, pumps, gas blown across the surface of the pond/bioreactor, and the movement associated with the addition of material at one end of the pond/bioreactor and removal at the other. Media components can be added at various points in the pond/bioreactor to provide different growing conditions for different phases of cell growth. Likewise, the temperature and pH of the culture can be varied at different points of the pond/bioreactor. Optionally, back mixing can be provided at various points. Active mixing can be achieved through the use of mixers, paddles, baffles or other appropriate techniques.

In a combination mode, a portion of the pond/bioreactor will operate in a plug flow mode, and a portion would operate in a stirred mode. For example, media can be added in a stirred zone to create a “self seeding” or “self inoculating” system. The media with growing cells would move from the stirred zone to a plug flow zone where the cells would continue their growth to the point of harvest. Stirred zones can be placed at the beginning, in the middle, or toward the end of the pond/bioreactor depending on the effect desired. In addition to creating a self seeding culture, such stirred zones can be used for purposes including, but not limited to, providing a specific residence time exposing the cells to specific conditions or concentrations of particular reagents or media components. Such stirred zones can be achieved through the use of baffles, barriers, diverters, and/or mixing devices.

A semi-continuous culture can be operated by charging the pond/bioreactor with an initial quantity of media and inoculums. As the growing continues, additional media is added either continuously, or at intervals.

In certain preferred embodiments, the algal culture may be grown in one or more closed (preferably sterilizable) bioreactors. Such closed culture and harvesting systems may be sterilized, thus greatly reducing problems from contaminating algae, bacteria, viruses and algae consuming micro-organisms and/or other extraneous species.

As used herein, “sterilization” includes any process that effectively kills or eliminates transmissible agents (such as fungi, bacteria, viruses, spore forms, etc.) from a surface, equipment, article of food or medication, or biological culture medium. Sterilization can be achieved through application of heat, chemicals, irradiation, high pressure, filtration, or combinations thereof. There are at least two broad categories of sterilization: physical and chemical. Physical sterilization includes: heat sterilization, radiation sterilization, high pressure gas sterilization (super critical CO₂). Chemical sterilization includes: ethylene oxide, ozone, chlorine bleach, glutaraldehyde formaldehyde, hydrogen peroxide, peracetic acid, or alcohol (e.g., 70% ethanol, 70% propanol), etc. Sterilization via radiation includes using ultraviolet (UV) light. All means described herein and those known in the art may be adapted for sterilizing the culture tanks, vessels, and containers used in the instant invention.

In certain embodiments, such bioreactors may be designed to be installed and operated in an outdoor environment, where it is exposed to environmental light and/or temperature. The apparatus, system and methods may be designed to provide improved thermal regulation useful for maintaining temperature within the range compatible with optimal growth and oil production. In certain embodiments, these systems may be constructed and operated on land that is marginal or useless for cultivation of standard agricultural crops, such as corn, wheat, soybeans, canola or rice.

In certain embodiments, the algae may be grown, at least during certain stages, in open ponds that may or may not be sterilizable. For example, in certain embodiments, a heterotrophic halophilic algae may be grown in the open air in a brine based medium, which conditions would substantially limit the growth of all other cells. Similarly, in certain embodiments, a thermophilic heterotrophic algae may be grown at a temperature that would limited growth of substantially all other organisms.

There is no particular limitation on the simplest apparatus for cultivating algae. However, the apparatus is preferably capable of supplying nutrients (including carbon dioxide) and light for autotrophic growth and, optionally, supplying nutrients (including organic carbon) for heterotrophic growth and, optionally, capable of irradiating a culture suspension with light under photoheterotrophic growth conditions. For example, in the case of a small-scale culture, a flat culture flask may be preferably used. In the case of a large-scale culture (such as culture in a race track or a channel-engineered system), a culture tank or vessel that is constituted by a transparent plate (e.g., made of glass, plastic, or the like) and that is equipped with an irradiation apparatus and an agitator, if necessary, may be used. Examples of such a culture tank include a plate culture tank, a tube-type culture tank, an airdome-type culture tank, and a hollow cylinder-type culture tank. In any case, a sealed container is preferably used.

Although natural lights may be used for autotrophic and photoheterotrophic growth, artificial light sources may also be used in the instant invention. In certain embodiments, guided light source (either natural or artificial in origin) may be used in the instant invention. For example, solar collectors may be used to gather natural sunlight, which in turn may be transmitted through a wave guide (e.g., fiber optic cables) to a specific site (bioreactor). A preferred artificial light source is LED, which provides one of the most efficient light energy source, since LED can provide light at a very specific wavelength that can be tailored for maximum cell utilization. In certain embodiments, LED emitting lights with a wavelength of about 400-500 nm, 400-460 nm, 620-680 nm, or 600-700 nm may be used.

Various carbon sources may be used for different stages of algal growth. For example, a simple sugar may be used as the carbon source. Alternatively, CO₂ may be used as the carbon source.

If CO₂ is used as the carbon source, it may be introduced into the closed system bioreactor, for example, by bubbling through the aqueous medium. In a preferred embodiment, CO₂ may be introduced by bubbling the gas through a perforated neoprene membrane, which produces small bubbles with a high surface to volume ratio for maximum exchange. In a more preferred embodiment, the gas bubbles may be introduced at the bottom of a water column in which the water flows in the opposite direction to bubble movement. This counterflow arrangement also maximizes gas exchange by increasing the time the bubbles are exposed to the aqueous medium. To further increase CO₂ dissolution, the height of the water column may be increased to lengthen the time that bubbles are exposed to the medium. The CO₂ dissolves in water to generate H₂CO₃, which may then be “fixed” by photosynthetic algae to produce organic compounds. Carbon dioxide can be supplied, for example, at a concentration of about 1-3% (v/v), at a rate of about 0.2-2 vvm. In other embodiments, higher CO₂ concentrations (e.g., up to 100%) and/or lower rate (e.g., less than 0.2 vvm) may also be used. When a plate culture tank is used, the culture suspension can also be stirred by supplying carbon dioxide, so that the algae (e.g., green algae) can be uniformly irradiated with light.

To switch the algal culture between different growing conditions, e.g., by exposing them to different types of plant growth regulators in a sequential manner, the algae can be physically harvested and separated from the medium. Harvest can occur directly from the pond or after transfer of the culture to a storage tank. The harvesting steps can include the steps of separating the cells from the bulk of the media, and/or re-using the medium for other batches of algal cultures.

Alternatively, switching medium can be effected by continuously diluting the algal culture growing under the first growing condition (e.g., first plant growth regulator) in a first bio-reactor, and collecting the displaced algal culture for growing in a second bio-reactor under the second growing condition (e.g., second plant growth regulator).

Another aspect of the invention is partly based on the discovery that certain plant growth regulators may be used to stimulate the production of certain algal products. Thus another aspect of the invention provides a method to produce an algal product, comprising culturing algae in the presence of a plant growth regulator or a mimic thereof to accumulate the algal product. In a preferred embodiment, the algal product is oil/lipid.

Preferably, for oil production, the plant growth regulator is an oil stimulating factor, such as a humate (e.g., fulvic acid, humic acid, or humin). The humate can be obtained from various sources, including commercial venders. In certain preferred embodiments, the following procedure may be used to produce the humate: about 25 g of powdered leonardite material (mined in Alberta, Canada, and supplied by Black Earth Humates Ltd, Edmonton, Alta., T5L 3C1) is hydrated with about 500 mL of a 1% NaOH solution. This is believed to release the combination of humic and fulvic acid into solution. After letting this mixture sit so that the organic ash material settles to the bottom, the liquid top portion is carefully drawn off. About 2 mL of 98% sulphuric acid is then added to acidify the drawn off portion. This is believed to cause the humic acid to precipitate to the bottom of the vessel. This portion is then divided between two 150 mL centrifuge containers. The two containers are then centrifuged for about 10 minutes at about 10,000 rpm. The humic acid is forced to the bottom, and the fulvic fraction is poured carefully off the top. Yield of the fulvic acid may vary, depending on the quality of the leonardite used. One of skill in the art can readily make minor variations of the method described herein without departing from the spirit of the invention.

In certain embodiments, the fulvic acid used is about 5-12.5% (v/v) of the growth medium.

According to this aspect of the invention, the primary purpose of growing algae is producing the desirable algal product. Thus, further algal cell number increase may waste valuable resource or energy, and is thus not desirable. Preferably, algal cell number increases by no more than one log (10-fold), 300%, 200%, 100%, or 50% under this growing condition.

Preferably, algal biomass substantially increases under the growing condition where bio-product accumulates. For example, algal biomass may increase largely as a result of accumulating the algal product. In certain embodiments, algal biomass increases by at least about 2-fold, 5-fold, 10-fold, 20-fold or 50-fold under such growing condition. For example, if the algal product proportion (e.g., oil, lipid, etc.) of the cell increases to 99% from 1%, a roughly 19-20 fold increase in algal biomass is achieved.

In certain embodiments, the accumulated algal product increases by at least about 10-fold, 20-fold, 50-fold, 100-fold, 200-fold, 500-fold, 1000-fold, 1500-fold, 2000-fold, 2500-fold or more under such growing condition. For example, if the algal product proportion (e.g., oil, lipid, etc.) of the cell increases to 99% from 1%, a roughly 1900 fold increase in algal product is achieved.

Preferably, the algae are also cultured in a nitrogen-limited medium or a medium with a nitrogen level optimized for algal product synthesis.

As described above, algae may be cultured in an open pond or in a bio-reactor, which may be adapted for optimal production of the algal product.

At the end of the growth period, algae can be recovered from the growing vessels (ponds and bioreactors). Separation of the cell mass from the bulk of the water/medium can be accomplished in a number of ways. Non-limiting examples include screening, centrifugation, rotary vacuum filtration, pressure filtration, hydrocycloning, flotation, skimming, sieving and gravity settling. Other techniques, such as addition of precipitating agents, flocculating agents, or coagulating agents, etc., can also be used in conjunction with these techniques. Two or more stages of separation can also be used. When multiple stages are used, they can be based on the same or a different technique. Non-limiting examples include screening of the bulk of the algal culture contents, followed by filtration or centrifugation of the effluent.

For example, algae may be partially separated from the medium using a standing whirlpool circulation, harvesting vortex and/or sipper tubes, as discussed below. Alternatively, industrial scale commercial centrifuges of large volume capacity may be used to supplement or in place of other separation methods. Such centrifuges may be obtained from known commercial sources (e.g., Cimbria Sket or IBG Monforts, Germany; Alfa Laval A/S, Denmark). Centrifugation, filtering, and/or sedimentation may also be of use to purify oil from other algal components. Separation of algae from the aqueous medium may be facilitated by addition of flocculants, such as clay (e.g., particle size less than 2 microns), aluminium sulphate or polyacrylamide. In the presence of flocculants, algae may be separated by simple gravitational settling, or may be more easily separated by centrifugation. Flocculent-based separation of algae is disclosed, for example, in U.S. Patent Appl. Publ. No. 20020079270, incorporated herein by reference.

The skilled artisan will realize that any method known in the art for separating cells, such as algae, from liquid medium may be utilized. For example, U.S. Patent Appl. Publ. No. 20040121447 and U.S. Pat. No. 6,524,486, each incorporated herein by reference, disclose a tangential flow filter device and apparatus for partially separating algae from an aqueous medium. Other methods for algal separation from medium have been disclosed in U.S. Pat. Nos. 5,910,254 and 6,524,486, each incorporated herein by reference. Other published methods for algal separation and/or extraction may also be used. See, e.g., Rose et al., Water Science and Technology 25: 319-327, 1992; Smith et al., Northwest Science 42: 165-171, 1968; Moulton et al., Hydrobiologia 204/205: 401-408, 1990; Borowitzka et al., Bulletin of Marine Science 47: 244-252, 1990; Honeycutt, Biotechnology and Bioengineering Symp. 13: 567-575, 1983.

Once the cell mass is harvested, the algal product (e.g., oil) can be liberated by disrupting (e.g., lysing) the algal cells using mechanical means, chemical (e.g., enzymatic) means, and/or solvent extraction.

Non-limiting examples of mechanical means for cell disruption include various types of presses, such as an expeller press, a batch press, a filter press, a cold press, a French press; pressure drop devices; pressure drop homogenizers, colloid mills, bead or ball mills, mechanical shearing devices (e.g., high shear mixers), thermal shock, heat treatment, osmotic shock, sonication or ultrasonication, expression, pressing, grinding, steam explosion, rotor-stator disruptors, valve-type processors, fixed geometry processors, nitrogen decompression or any other known method. High capacity commercial cell disruptors may be purchased from known sources. (E.g., GEA Niro Inc., Columbia, Md.; Constant Systems Ltd., Daventry, England; Microfluidics, Newton, Mass.). Methods for rupturing microalgae in aqueous suspension are disclosed, for example, in U.S. Pat. No. 6,000,551, incorporated herein by reference.

Non-limiting examples of chemical means include the use of enzymes, oxidizing agents, solvents, surfactants, and chelating agents. Depending on the exact nature of the technique being used, the disruption can be done dry, or a solvent, water, or steam can be present.

Solvents that can be used for the disrupting or to assist in the disrupting include, but are not limited to hexane, heptane, alcohols, supercritical fluids, chlorinated solvents, alcohols, acetone, ethanol, methanol, isopropanol, aldehydes, ketones, chlorinated solvents, fluorinated-chlorinated solvents, and combinations thereof. Exemplary surfactants include, but are not limited to, detergents, fatty acids, partial glycerides, phospholipids, lysophospholipids, alcohols, aldehydes, polysorbate compounds, and combinations thereof. Exemplary supercritical fluids include carbon dioxide, ethane, ethylene, propane, propylene, trifluoromethane, chlorotrifluoromethane, ammonia, water, cyclohexane, n-pentane, and toluene. The supercritical fluid solvents can also be modified by the inclusion of water or some other compound to modify the solvent properties of the fluid. Suitable enzymes for chemical disrupting include proteases, cellulases, lipases, phospholipases, lysozyme, polysaccharases, and combinations thereof. Suitable chelating agents include, but are not limited to EDTA, porphine, DTPA, NTA, HEDTA, PDTA, EDDHA, glucoheptonate, phosphate ions (variously protonated and nonprotonated), and combinations thereof. In some cases, solvent extraction can be combined with mechanical or chemical cell disrupting as described herein. Combinations of chemical and mechanical methods can also be used.

Separation of the broken cells from the product containing portion or phase can be accomplished by various techniques. Non-limiting examples include centrifugation, hydrocycloning, filtration, floatation, and gravity settling. In some situations, it would be desirable to include a solvent or supercritical fluid, for example, to solubilize desired products, reduce interaction between the product and the broken cells, reduce the amount of product remaining with the broken cells after separation, or to provide a washing step to further reduce losses. Suitable solvents for this purpose include, but are not limited to hexane, heptane, supercritical fluids, chlorinated solvents, alcohols, acetone, ethanol, methanol, isopropanol, aldehydes, ketones, and fluorinated-chlorinated solvents. Exemplary supercritical fluids include carbon dioxide, ethane, ethylene, propane, propylene, trifluoromethane, chlorotrifluoromethane, ammonia, water, cyclohexane, n-pentane, toluene, and combinations of these. The supercritical fluid solvents can also be modified by the inclusion of water or some other compound to modify the solvent properties of the fluid.

The product so isolated can then be further processed as appropriate for its desired use such as by solvent removal, drying, filtration, centrifugation, chemical modification, transesterification, further purification, or by some combination of steps.

For example, lipids/oils can be isolated from the biomass and then used to form biodiesel using methods known to form biodiesel. For example, the biomass can be pressed and the resulting lipid-rich liquid separated, using any of the methods described herein. The separated oil can then be processed into biodiesel using standard transesterification technologies, such as the well-known Connemann process (see, e.g., U.S. Pat. No. 5,354,878, the entire text of which is incorporated herein by reference).

For example, the algae may be harvested, separated from the liquid medium, disrupting and the oil content separated (supra). The algal-produced oil will be rich in triglycerides. Such oils may be converted into biodiesel using well-known methods, such as the Connemann process (see, e.g., U.S. Pat. No. 5,354,878, incorporated herein by reference), which is well-established for production of biodiesel from plant sources such as rapeseed oil. Standard transesterification processes involve an alkaline catalyzed transesterification reaction between the triglyceride and an alcohol, typically methanol. The fatty acids of the triglyceride are transferred to methanol, producing alkyl esters (biodiesel) and releasing glycerol. The glycerol is removed and may be used for other purposes.

In contrast to batch reaction methods (e.g., J. Am. Oil Soc. 61: 343, 1984), the Connemann process utilizes continuous flow of the reaction mixture through reactor columns, in which the flow rate is lower than the sinking rate of glycerine. This results in the continuous separation of glycerine from the biodiesel. The reaction mixture may be processed through further reactor columns to complete the transesterification process. Residual methanol, glycerine, free fatty acids and catalyst may be removed by aqueous extraction.

However, the skilled artisan will realize that any method known in the art for producing biodiesel from triglyceride containing oils may be utilized, for example, as disclosed in U.S. Pat. Nos. 4,695,411; 5,338,471; 5,730,029; 6,538,146; 6,960,672, each incorporated herein by reference. Alternative methods that do not involve transesterification may also be used. For example, by pyrolysis, gasification, or thermochemical liquefaction (see, e.g., Dote, Fuel 73: 12, 1994; Ginzburg, Renewable Energy 3: 249-252, 1993; Benemann and Oswald, DOE/PC/93204-T5, 1996).

Although there are thousands of species of known, naturally occurring algae, many (if not most) may be used for oil/lipid/biodiesel production and formation of other products. These algae may be metabolizing under heterotrophic, photoheterotrophic, or autotrophic conditions. Particularly preferred algae that may be used for the instant invention include Chlorophytes or Bacilliarophytes (diatoms).

In certain embodiments, the algae may be genetically modified/engineered to further increase biodiesel feedstock production per unit acre. The genetic modification of algae for specific product outputs is relatively straight forward using techniques well known in the art. However, the low-cost methods for cultivation, harvesting, and product extraction disclosed herein may be used with genetically modified (e.g., transgenic, non-transgenic) algae. The skilled artisan will realize that different algal strains will exhibit different growth and oil productivity and that under different conditions, the system may contain a single strain of algae or a mixture of strains with different properties, or strains of algae plus symbiotic bacteria. The algal species used may be optimized for geographic location, temperature sensitivity, light intensity, pH sensitivity, salinity, water quality, nutrient availability, seasonal differences in temperature or light, the desired end products to be obtained from the algae and a variety of other factors.

In certain embodiments, algae of use to produce the bio-product (e.g., oil/biodiesel) may be genetically engineered (e.g., transgenic or generated by site directed mutagenesis, etc.) to contain one or more isolated nucleic acid sequences that enhance bio-product production or provide other characteristics of use for algal culture, growth, harvesting or use. Methods of stably transforming algal species and compositions comprising isolated nucleic acids of use are well known in the art and any such methods and compositions may be used in the practice of the present invention. Exemplary transformation methods of use may include microprojectile bombardment, electroporation, protoplast fusion, PEG-mediated transformation, DNA-coated silicon carbide whiskers or use of viral mediated transformation (see, e.g., Sanford et al., 1993, Meth. Enzymol. 217:483-509; Dunahay et al., 1997, Meth. Molec. Biol. 62:503-9; U.S. Pat. Nos. 5,270,175; 5,661,017, incorporated herein by reference).

For example, U.S. Pat. No. 5,661,017 discloses methods for algal transformation of chlorophyll C-containing algae, such as the Bacillariophyceae, Chrysophyceae, Phaeophyceae, Xanthophyceae, Raphidophyceae, Prymnesiophyceae, Cryptophyceae, Cyclotella, Navicula, Cylindrotheca, Phaeodactylum, Amphora, Chaetoceros, Nitzschia or Thalassiosira. Compositions comprising nucleic acids of use, such as acetyl-CoA carboxylase, are also disclosed.

In various embodiments, a selectable marker may be incorporated into an isolated nucleic acid or Vector to select for transformed algae. Selectable markers of use may include neomycin phosphotransferase, aminoglycoside phosphotransferase, aminoglycoside acetyltransferase, chloramphenicol acetyl transferase, hygromycin B phosphotransferase, bleomycin binding protein, phosphinothricin acetyltransferase, bromoxynil nitrilase, glyphosate-resistant 5-enolpyruvylshikimate-3-phosphate synthase, cryptopleurine-resistant ribosomal protein S14, emetine-resistant ribosomal protein S14, sulfonylurea-resistant acetolactate synthase, imidazolinone-resistant acetolactate synthase, streptomycin-resistant 16S ribosomal RNA, spectinomycin-resistant 16S ribosomal RNA, erythromycin-resistant 23S ribosomal RNA or methyl benzimidazole-resistant tubulin. Regulatory nucleic acid sequences to enhance expression of a transgene are known, such as C. cryptica acetyl-CoA carboxylase 5′-untranslated regulatory control sequence, a C. cryptica acetyl-CoA carboxylase 3′-untranslated regulatory control sequence, and combinations thereof.

### Example 1

Chlorella vulgaris was cultured in Bristol's medium (see Nichols, Growth Media—freshwater. In: Psychological Methods. Ed. J. R. Stern. Cambridge University Press, Pp 7-24, 1973, incorporated by reference; also see below in Table 1), amended with 0.1% yeast extract (DIFCO, MI—Bacto Yeast Extract, product number 212750) and 0.5% glucose (control cells). A second group was cultured in the same medium with a 10% addition of fulvic acid, which was extracted from leonardite (20-25% fulvic acid).

TABLE 1
Autotrophic and Heterotrophic Bristol's Media (mg/L)

| Chemical | Autotrophic | Heterotrophic |
|---|---|---|
| NaNO₃ | 250 | 250 |
| CaCl₂•2H₂O | 25 | 25 |
| MgSO₄•7H₂O | 75 | 75 |
| K₂HPO₄ | 75 | 75 |
| KH₂PO₄ | 175 | 175 |
| NaCl | 25 | 25 |
| EDTA | 50 | 50 |
| KOH | 31 | 31 |
| Fe₂SO₄•7H₂O | 4.98 | 4.98 |
| H₂SO₄ | 0.001 mL/L | 0.001 mL/L |
| H₃BO₃ | 11.42 | 11.42 |
| ZnSO₄•7H₂O | 8.82 | 8.82 |
| MnCl₂•4H₂O | 1.44 | 1.44 |
| MoO₃ | 0.71 | 0.71 |
| CuSO₄•5H₂O | 1.57 | 1.57 |
| Co(NO₃)₂•6H₂O | 0.49 | 0.49 |
| Yeast Extract | — | 1000 |
| C₆H₁₂O₆ | — | 5000 |

Stock Solutions can be made for easy addition of the chemicals to the media.

To prepare the fulvic acid, about 25 g of powdered leonardite material (mined in Alberta, Canada, and supplied by Black Earth Humates Ltd, Edmonton, Alta., T5L 3C1) was hydrated with about 500 mL of a 1% NaOH solution. This is believed to release the combination of humic and fulvic acid into solution. After letting this mixture sit so that the organic ash material settled to the bottom, the liquid top portion was carefully drawn off. About 2 mL of 98% sulphuric acid was then added to acidify the drawn off portion. This is believed to cause the humic acid to precipitate to the bottom of the vessel. This portion was then divided between two 150 mL centrifuge containers. The two containers were then centrifuged for about 10 minutes at about 10,000 rpm. The humic acid was forced to the bottom, and the fulvic fraction was poured carefully off the top. Yield of the fulvic acid may vary, depending on the quality of the leonardite used. Typically, using the current material yields approximately 250-280 mL of the fulvic acid fraction. This fulvic acid was then used at a rate between 5-12.5% (v/v) of the growth medium.

The control cells had an average radius of about 3.4 m with minimal vacuole development. The cells cultured in the medium amended with fulvic acid had a wide diversity of cell sizes. The large cells reached an average radius of about 5.6 m and exhibited very large vacuoles. These vacuoles were lipid-containing, as confirmed using Nile Red staining. The fulvic acid stimulated the cells to produce storage products far in excess of the control cells.

Notably, in the example shown herein, a significant number of algal cells were induced into storage mode in the presence of fulvic acid, despite the fact that the nitrogen in the medium was non-limiting. It is expected that a substantial increase in frequency of the large lipid vacuole-containing cells will occur when the algal cells, are cultured under conditions with limited nitrogen. In addition, it is expected that the oil content in the culture will be well into the 80+% (probably 90+%) range.

### Example 2

Auxenochlorella protothecoides was grown in Bristol's medium (see above) amended with 0.1% yeast extract (see above) and 0.5% glucose (control cells). Two other groups were cultured in the same medium with either indole acetic acid (2 mg/L, Cat. No. 12886, Sigma-Aldrich Canada Ltd.) or gibberellic acid (2 mg/L, Cat. No. G7645, Sigma-Aldrich Canada Ltd.) added. Dry weights were determined and compared between the culture groups after seven days.

Those treated with indole acetic acid increased dry cell mass by 50% relative to the control. Those treated with gibberellic acid increased dry cell mass by 20%. Further, those cells treated with indole acetic acid increased oil production by 15%.

### Example 3

### Comparison of the Growth of Chlorella protothecoides with or without Certain Combination of Growth Factors

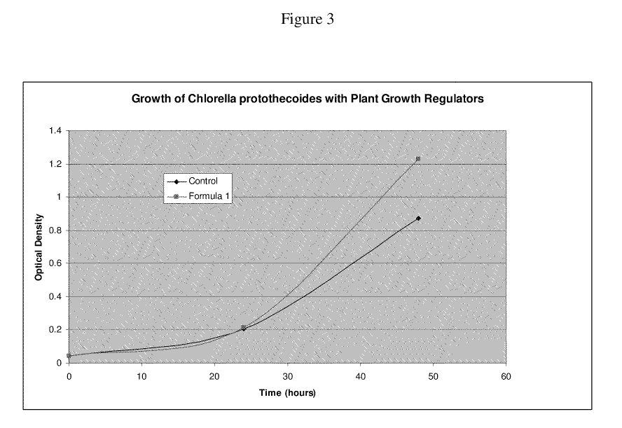

The stock formula used was 0.25 g kinetin, 0.25 g 6-BA, 0.5 g NAA, 0.5 g GA3, 1.0 g Vitamin B1, 1.0 L dH₂O. 19.5 mL were added to 250 mL of HGM (see table below) to create formula 2. Flasks were inoculated with Chlorella protothecoides to give a starting optical density of 0.04 absorbance units. The flasks were placed on a shaker at 125 rpm under heterotrophic (dark) conditions. Temperature was maintained at about 23° C. Optical densities were measured daily. Results are summarized in FIG. 3.

TABLE 2
Heterotrophic Growth Medium (HGM)

| | | | | Stock | |
|---|---|---|---|---|---|
| | | | | Solution | |
| Stock | | Amount | | Conc. | Final |
| Solution | Component | (L⁻¹) | | (400 mL⁻¹) | Concentration |
| 1 | NaNO₃ | 30 | ml | 10 g | 8.82 mM |
| 2 | CaCl₂•(2H₂O) | 30 | ml | 1 g | 0.17 mM |
| 3 | MgSO₄•(7H₂O) | 30 | ml | 3 g | 0.30 mM |
| 4 | K₂HPO₄ | 30 | ml | 3 g | 0.43 mM |
| 5 | KH₂PO₄ | 30 | ml | 7 g | 1.29 mM |
| 6 | NaCl | 30 | ml | 1 g | 0.43 mM |
| 7 | Trace Metal (sol) | 18 | ml | See note 1 | |
| 8 | Yeast Extract (Bacto) | 4 | g | NA | 0.4% |
| 9 | C₆H₁₂O₆ | 20 | g | NA | 2.0% |

Note 1:
NaEDTA•2H₂O, 075 g/L; FeCl₃•6H₂O, 0.097 g/L; MgCl₂•4H₂O, 0.041 g/L; boric acid, 0.011 g/L; ZnCl₂, 0.005 g/L; CoCl₂•6H₂O, 0.002 g/L; CuSO₄, 0.002 g/L; Na₂MoO₄•H₂O, 0.002 g/L.
Note 2:
the HGM is a modified Bristol's medium with increased NaNO₃ concentration (from 2.94 mM final concentration to 8.82 mM final concentration), and additional components, including 0.4% Yeast Extract (Bacto), 2.0% glucose, and a mixture of trace metals (see Note 1). Glucose is absent in the traditional Bristol's medium because algae growing under phototrophic conditions use photosynthesis to produce organic compounds such as carbon hydrates.
Note 3:
Medium was placed in Nephelo flasks (250 ml) and sterilized at 121° C. for 20 minutes.

It was shown that Formula 1 generated biomass at a faster rate than did the control heterotrophic growth medium. The specific growth rates, μ, were 1.4 and 1.8 for the control and Formula 1, respectively.

### Example 4

### Comparison of the Growth of Chlorella protothecoides with or without Certain Combination of Growth Factors

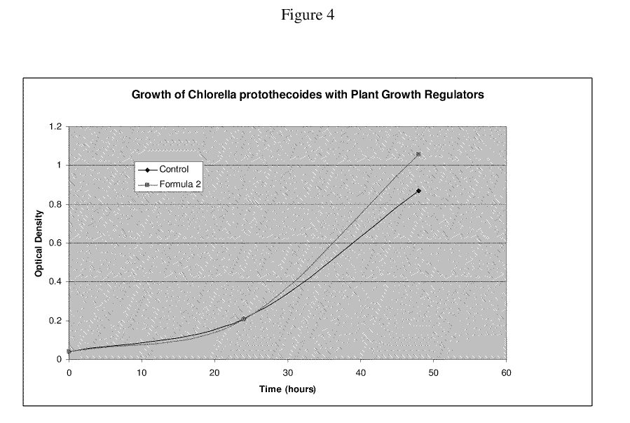

The stock formula used was 0.25 g kinetin, 0.25 g 6BA, 0.5 g NAA, 0.5 g GA3, 1.0 g Vitamin B1, 1.0 L dH₂O. 4.7 nL were added to 250 mL of HGM (see table above) to create formula 2. Flasks were inoculated with Chlorella protothecoides to give a starting optical density of 0.04 absorbance units. The flasks were placed on a shaker at 125 rpm under heterotrophic (dark) conditions. Temperature was maintained at about 23° C. Optical densities were measured daily. Results are summarized in FIG. 4.

It was shown that formula 2 generated biomass at a faster rate than did the control heterotrophic growth medium. The specific growth rates, μ, were 1.4 and 1.6 for the control and formula 2, respectively.

### Example 5

### Comparison of the Growth of Chlorella protothecoides with or without Certain Combination of Growth Factors

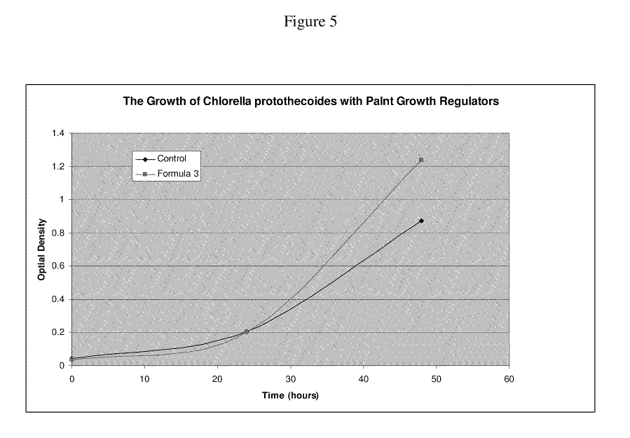

The stock formula used was 0.25 g kinetin, 0.25 g 6BA, 0.25 g NAA, 0.25 g IAA, 0.5 g GA3, 1.0 g Vitamin B1, 1.0 L dH₂O. 19.5 nil were added to 250 mL of HGM (see table above) to create formula 3. Flasks were inoculated with Chlorella protothecoides to give a starting optical density of 0.04 absorbance units. The flasks were placed on a shaker at 125 rpm under heterotrophic (dark) conditions. Temperature was maintained at about 23° C. Optical densities were measured daily. Results are summarized in FIG. 5.

In was shown that formula 3 generated biomass at a faster rate than did the control heterotrophic growth medium. The specific growth rates, μ, were 1.4 and 1.8 for the control and formula 3, respectively.

### Example 6

### Comparison of the Growth of Chlorella protothecoides with or without Certain Combination of Growth Factors

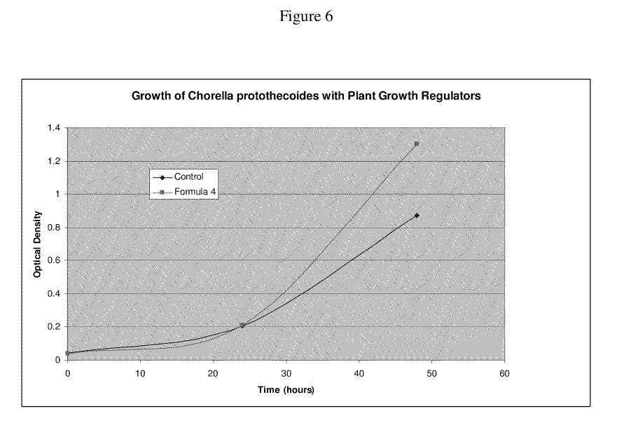

The stock formula used was 0.25 g kinetin, 0.25 g 6BA, 0.25 g NAA, 0.25 g IAA, 0.5 g GA3, 1.0 g Vitamin B1, 1.0 L dH₂O. 4.7 nL were added to 250 mL of HGM (see table above) to create formula 4. Flasks were inoculated with Chlorella protothecoides to give a starting optical density of 0.04 absorbance units. The flasks were placed on a shaker at 125 rpm under heterotrophic (dark) conditions. Temperature was maintained at about 23° C. Optical densities were measured daily. Results are summarized in FIG. 6.

It was shown that formula 4 generated biomass at a faster rate than did the control heterotrophic growth medium. The specific growth rates, μ, were 1.4 and 1.8 for the control and formula 4, respectively.

The regulator concentrations used above are summarized in Table 3 below.

TABLE 3
Summary of Plant Growth Regulator Stimulated Algal Growth

| | | | | | | | | Control | Exp. |
|---|---|---|---|---|---|---|---|---|---|
| Kinetin | 6BA | NAA and/or | | GA3 | Vitamin | Stock Vol. | | Growth | Growth |
| (L⁻¹) | (L⁻¹) | IAA (L⁻¹) | | (L⁻¹) | B1 (L⁻¹) | per flask | | Rate (μ) | Rate (μ) |
| 0.25 g | 0.25 g | 0.5 | g NAA | 0.5 g | 1.0 g | 19.5 | nL | 1.4 | 1.8 |
| 0.25 g | 0.25 g | 0.5 | g NAA | 0.5 g | 1.0 g | 4.7 | nL | 1.4 | 1.6 |
| 0.25 g | 0.25 g | 0.25 | g NAA; | 0.5 g | 1.0 g | 19.5 | nL | 1.4 | 1.8 |
| | | 0.25 | g IAA | | | | | | |
| 0.25 g | 0.25 g | 0.25 | g NAA; | 0.5 g | 1.0 g | 4.7 | nL | 1.4 | 1.8 |
| | | 0.25 | g IAA | | | | | | |

### Example 7

### Photoheterotropic and Heterotrophic Growth

The influence of light exposure during Scenedesmus obliquus and Chlorella protothecoides growth was assessed. The growth rates of both algae were higher in photoheterotrophic growth conditions. The Scenedesmus obliquus growth rate was about 86.7% higher under photoheterotrophic growth. Meanwhile, the Chlorella protothecoides growth rate increased 39.07% when the growth was conducted under photoheterotrophic growth. The results of these experiments are summarized in Tables 4-7 below.

TABLE 4
The effect of different hormone concentrations on growth rate of Scenedesmus obliquus
cultured in photoheterotrophic conditions for 48 hours

| Hormones | 100 ng | 10 ng | 1 ng | 0.1 ng | 0.01 ng |
|---|---|---|---|---|---|
| Indole-3- | 0.62 ± 0.092 | 0.49 ± 0.023 | 0.49 ± 0.030 | 0.47 ± 0.061 | 0.42 ± 0.020 |
| acetic acid | | | | | |
| 1-Naphthalene- | 0.73 ± 0.046 | 0.80 ± 0.141 | 0.81 ± 0.042 | 0.85 ± 0.042 | 0.84 ± 0.087 |
| acetic acid | | | | | |
| 2,4-Dichloro- | 0.33 ± 0.042 | 0.44 ± 0.028 | 0.47 ± 0.023 | 0.44 ± 0.000 | 0.42 ± 0.035 |
| phenoxyacetic | | | | | |
| Kinetin | 0.36 ± 0.060 | 0.37 ± 0.070 | 0.92 ± 0.113 | 0.73 ± 0.042 | 0.57 ± 0.133 |
| 6-Benzyl- | 0.52 ± 0.060 | 0.47 ± 0.064 | 0.47 ± 0.011 | 0.37 ± 0.099 | 0.46 ± 0.056 |
| aminopurine | | | | | |
| Gibberellic | 0.51 ± 0.110 | 0.56 ± 0.141 | 0.56 ± 0.087 | 0.47 ± 0.081 | 0.59 ± 0.064 |
| acid | | | | | |

| | |
|---|---|
| Control | 0.41 ± 0.042 |

TABLE 5
The effect of different hormone concentrations on growth rate of Scenedesmus obliquus
cultured in heterotrophic conditions for 48 hours

| Hormones | 100 ng | 10 ng | 1 ng | 0.1 ng | 0.01 ng |
|---|---|---|---|---|---|
| Indole-3- | 0.41 ± 0.053 | 0.47 ± 0.020 | 0.42 ± 0.081 | 0.36 ± 0.127 | 0.23 ± 0.020 |
| acetic acid | | | | | |
| 1-Naphthalene- | 0.39 ± 0.053 | 0.28 ± 0.099 | 0.33 ± 0.020 | 0.28 ± 0.011 | 0.26 ± 0.042 |
| acetic acid | | | | | |
| 2,4-Dichloro- | 0.23 ± 0.040 | 0.24 ± 0.081 | 0.31 ± 0.020 | 0.23 ± 0.040 | 0.28 ± 0.030 |
| phenoxyacetic | | | | | |
| Kinetin | 0.28 ± 0.076 | 0.31 ± 0.028 | 0.36 ± 0.042 | 0.26 ± 0.076 | 0.28 ± 0.061 |
| 6-Benzyl- | 0.33 ± 0.104 | 0.36 ± 0.092 | 0.39 ± 0.092 | 0.32 ± 0.061 | 0.28 ± 0.081 |
| aminopurine | | | | | |
| Gibberellic | 0.42 ± 0.064 | 0.36 ± 0.050 | 0.43 ± 0.020 | 0.50 ± 0.046 | 0.44 ± 0.083 |
| acid | | | | | |

| | |
|---|---|
| Control | 0.35 ± 0.023 |

TABLE 6
The effect of different hormone concentrations on growth rate of
Chlorella protothecoides cultured in photoheterotrophic conditions for 48 hours

| Hormones | 100 ng | 10 ng | 1 ng | 0.1 ng | 0.01 ng |
|---|---|---|---|---|---|
| Indole-3- | 1.02 ± 0.061 | 1.13 ± 0.019 | 0.97 ± 0.020 | 1.05 ± 0.019 | 1.06 ± 0.030 |
| acetic acid | | | | | |
| 1-Naphthalene- | 1.16 ± 0.152 | 1.07 ± 0.028 | 1.05 ± 0.035 | 1.02 ± 0.050 | 1.00 ± 0.058 |
| acetic acid | | | | | |
| 2,4-Dichloro- | 1.03 ± 0.069 | 1.08 ± 0.030 | 1.01 ± 0.035 | 1.08 ± 0.133 | 1.09 ± 0.035 |
| phenoxyacetic | | | | | |
| Kinetin | 1.19 ± 0.035 | 1.18 ± 0.050 | 1.02 ± 0.011 | 1.10 ± 0.042 | 1.08 ± 0.023 |
| 6-Benzyl- | 1.08 ± 0.023 | 1.04 ± 0.083 | 1.07 ± 0.035 | 1.12 ± 0.011 | 1.00 ± 0.030 |
| aminopurine | | | | | |
| Gibberellic | 1.10 ± 0.070 | 1.09 ± 0.122 | 1.00 ± 0.030 | 1.02 ± 0.046 | 1.06 ± 0.011 |
| acid | | | | | |

| | |
|---|---|
| Control | 1.05 ± 0.020 |

TABLE 7
The effect of different hormone concentrations on growth rate of
Chlorella protothecoides cultured in heterotrophic conditions for 48 hours

| Hormones | 100 ng | 10 ng | 1 ng | 0.1 ng | 0.01 ng |
|---|---|---|---|---|---|
| Indole-3- | 1.60 ± 0.076 | 1.60 ± 0.099 | 1.49 ± 0.122 | 1.61 ± 0.072 | 1.62 ± 0.133 |
| acetic acid | | | | | |
| 1-Naphthalene- | 1.62 ± 0.064 | 1.57 ± 0.028 | 1.62 ± 0.136 | 1.54 ± 0.081 | 1.66 ± 0.140 |
| acetic acid | | | | | |
| 2,4-Dichloro- | 1.50 ± 0.081 | 1.31 ± 0.087 | 1.43 ± 0.069 | 1.53 ± 0.069 | 1.40 ± 0.061 |
| phenoxyacetic | | | | | |
| Kinetin | 1.58 ± 0.061 | 1.60 ± 0.070 | 1.44 ± 0.110 | 1.50 ± 0.050 | 1.60 ± 0.050 |
| 6-Benzyl- | 1.46 ± 0.150 | 1.52 ± 0.117 | 1.50 ± 0.012 | 1.54 ± 0.081 | 1.48 ± 0.121 |
| aminopurine | | | | | |
| Gibberellic | 1.46 ± 0.050 | 1.52 ± 0.099 | 1.46 ± 0.090 | 1.52 ± 0.151 | 1.52 ± 0.201 |
| acid | | | | | |

| | |
|---|---|
| Control | 1.54 ± 0.080 |

1. A method to increase cell proliferation of algae, comprising culturing the algae in the presence of one or more plant growth regulator, mimics thereof, or mixtures thereof to increase algal cell number.

2-4. (canceled)

5. The method of claim 1, wherein the plant growth regulator comprises at least one, two, three, four, five, or more growth hormones selected from: an Auxin, a Cytokinin, a Gibberellin, and/or a mixture thereof.

6. The method of claim 5, wherein the Auxin comprises indole acetic acid (IAA) and/or 1-Naphthaleneacetic acid (NAA).

7. The method of claim 5, wherein the Gibberellin comprises GA3.

8. The method of claim 5, wherein the Cytokinin is an adenine-type cytokinin or a phenylurea-type cytokinin.

9. The method of claim 8, wherein the adenine-type cytokinin comprises kinetin, zeatin, and/or 6-benzylaminopurine, and the phenylurea-type cytokinin comprises diphenylurea and/or thidiazuron (TDZ).

10. The method of claim 1, wherein the plant growth regulator further comprises vitamin B1 or analog/mimics thereof.

11. The method of claim 5, wherein the ratio (w/w) of Auxin to Cytokinin is about 1:2 to 2:1 (w/w), or about 1:1 (w/w).

12. The method of claim 5, wherein the ratio (w/w) of Auxin to Gibberellin is about 1:2 to 2:1 (w/w), or about 1:1 (w/w).

13. The method of claim 1, wherein the mimic is a phenoxyacetic compound.

14-20. (canceled)

21. The method of claim 1, wherein the algae metabolize using heterotrophic, photoheterotrophic, or autotrophic physiological mechanisms.

22. The method of claim 1, wherein the algae are Chromophytes.

23. The method of claim 1, wherein the algae are Chlorophytes or Bacillariophytes.

24. The method of claim 1, wherein the algae have frustule free forms.

25-26. (canceled)

27. A method to produce an algal product, comprising culturing algae in the presence of a plant growth regulator or a mimic thereof to accumulate the algal product.

28-31. (canceled)

32. The method of claim 27, wherein the algae is cultured in a nitrogen-limited medium or a medium with a nitrogen level optimized for algal product synthesis.

33. The method of claim 27, wherein the plant growth regulator comprises an oil stimulating factor.

34. The method of claim 33, wherein the oil stimulating factor comprises a humate, such as fulvic acid or humic acid.

35-37. (canceled)

38. The method of claim 27, wherein the algal product is oil or lipid.

39. The method of claim 38, wherein the algal product comprises Omega-3, -6, and/or -9.
